(19) United States
(12) Patent Application Publication
(10) Pub. No.: US 2006/0270040 A1
(43) Pub. Date: Nov. 30, 2006

(54) Compositions and methods for treating tissue

(75) Inventors: Marcin Filutowicz, Madison, WI (US); Hideki Suzuki, Madison, WI (US)

Correspondence Address: Medlen & Carroll, LLP;Suite 350, 101 Howard Street, San Francis, CA, 94105, US

(73) Assignee: ConjuGon, Inc., Madison, WI (US)

(21) Appl. No.: 11/137,950

(22) Filed: May 26, 2005

(51) Int. Cl. C12N 15/74; C12N 1/21; C12N 5/06

(52) U.S. Cl. 435/455; 435/471; 435/252.3; 435/325

(57) ABSTRACT

The present invention relates to the field of bacteriology. In particular, the invention relates to novel compositions and methods for altering (e.g., inhibiting) the growth and virulence of populations of pathogenic microorganisms.

### FIELD OF THE INVENTION

The present invention relates to the field of bacteriology. In particular, the invention relates to novel compositions (e.g., antimicrobial agents) and methods of using the same for treating tissue (e.g., lesions of the skin and other soft-tissues). In some embodiments, the present invention comprises the killing or altering (e.g., inhibiting) growth and virulence of populations of microorganisms.

### BACKGROUND OF THE INVENTION

The spread of antibiotic resistant pathogens such as methicillin-resistant Staphylococcus aureus (MRSA) and macrolide-resistant Streptococcus pyogenes, and multi-drug-resistant Pseudomonas aeruginosa have made the treatment of skin and soft-tissue infections increasingly difficult (Fung et al., Drugs 63: 1459-80 (2003)).

For example, one of the persistent problems of bum wound care is the development of microbial infections. Humans live not in a sterile environment but in a symbiotic relationship with bacteria and other microbes. The intact skin and mucosal surface act to maintain a delicate balance between our tissues and the bacterial populations. Any breach in the skin or mucosal barriers alters this balance and thus has the potential to initiate infections by allowing bacteria to gain access to the underlying tissues and achieve critical numbers. One of the major treatment goals of a bum surgeon is to prevent infections and, if contamination occurs, the goal is to reduce the microbial contamination below the critical numbers required to initiate and spread infections. With the discovery of antibiotics, bum wound infections appeared to be under control. However, it has been discovered that bacteria are able to, and indeed have, overcome the antibiotics through development of resistance. Emergence of resistant strains of bacteria has become the major source of many hospital-based infections and has posed a major clinical dilemma to burn surgeons.

The problem of antibiotic resistance affects all kinds of bacterial infections, including but not limited to infections of the skin and soft-tissues. There are many examples of the rampant rise in antibiotic resistance in pathogenic organisms. In one hospital in Corpus Christi, Tex., community-acquired methicillin-resistant Staphylococcus aureus (MRSA), most often seen in skin and soft-tissue infections, slowly increased from 3% in 1990 to 10% in 1999 and then rapidly increased over a 4-year period to 62% in 2003 (Goodman, J Clin Invest 114, 1181 (2004)). In a Miami hospital, P. aeruginosa resistance to quinolones in leg ulcers increased from 19% in 1992 to 56% in 2001 (Valencia et al., J Am Acad Dermatol 50, 845-9 (2004)). There is no debate in the field, new treatments are needed to control these infections.

Prophylactic use of antibacterial agents such as silver nitrate, silver sulfadiazine (Silverdene, Thermazine, Flamazine) and mafenide acetate (Sulfamylon) has become the standard of care to reduce bacteria colonization in wounds such as burn wounds. However, these agents have limitations. For example, Silverdene has been shown to retard wound healing and cannot be used in patients who are allergic to sulfa drugs. The metabolic products of Sulfamylon are potent inhibitors of carbonic anhydrase and therefore can cause metabolic acidosis. Use of this compound is particularly contraindicated in patients who have suffered inhalation injury and those who developed sepsis.

Other antimicrobial agents that are used to prevent or reduce bacterial colonization are Gentamicin sulfate, Bacitracin, Nitrofurantoin. Unfortunately constant use of these antimicrobial agents results in the emergence of resistant strains of the offending bacteria.

Despite the acceptance of these antimicrobial strategies as standard of care in the treatment of burn patients, development of drug resistant bacterial infections (e.g., methicillin resistant Staphylococcus aureus, Pseudomonas aeruginosa and Acinetobacter baumannii) continue to pose significant clinical problems in patients (e.g., critically injured burn patients or diabetic patients with chronic ulcers) during prolonged hospitalization.

Thus, a great need exists to develop alternative strategies of antimicrobial treatment. In particular, treatments are needed that can address and effectively kill or attenuate drug resistant microorganisms.

### SUMMARY OF THE INVENTION

The present invention relates to the field of bacteriology. In particular, the invention relates to novel compositions (e.g., antimicrobial agents) and methods of using the same for treating tissue (e.g., lesions of the skin and other soft tissues) comprising the killing or altering (e.g., inhibiting) growth and virulence of populations of microorganisms.

In some embodiments, the methods of the present invention comprises treating a tissue by exposing the tissue to a donor cell, wherein said donor cell comprises a recombinant transmissible plasmid comprising a gene encoding a bactericidal protein and a helper plasmid comprising a gene encoding an immunity protein, wherein said immunity protein is configured to inhibit said bactericidal protein. In such embodiments, the donor cell is configured to conjugatively transfer the recombinant transmissible plasmid to a recipient cell, such that the recombinant transmissible plasmid expresses the gene encoding a bactericidal protein in the recipient cell. In preferred embodiments, expression of the gene encoding a bactericidal protein is lethal to the recipient cell. In some embodiments, the bactericidal protein is a colicin. In some preferred embodiments, the colicin is colE3, while in other preferred embodiments, the bactericidal protein includes but is not limited to colA, colB, colD, colIa, colIb, colK, colN, colE1, colE2, colE4, colE5, colE6, colE7, colE8, colE9, or lysozyme.

The methods of the present invention contemplate the use of an immunity protein configured to inhibit the effects of the bactericidal protein. In preferred embodiments, the immunity protein binds to the bactericidal protein. For example, the immunity protein immE3 binds to and inhibits (e.g., inactivates) the bactericidal protein colE3. Numerous pairs of bactericidal proteins and corresponding immunity proteins are known in the art. In the present invention, the bactericidal proteins listed above are inhibited by the corresponding colicin A, colicin B, colicin D, colicin Ia, colicin Ib, colicin K, colicin N, colicin E1, colicin E2, colicin E4, colicin E5, colicin E6, colicin E7, colicin E8, and colicin E9 immunity proteins, respectively.

The present invention provides compositions and methods for treating tissue, including but not limited to skin, mucosal tissue, lung tissue, bladder tissue, etc. In some embodiments, undamaged tissue is treated, while in some embodiments, the tissue comprises a wound. In some preferred embodiments, the wound comprises a bum wound.

In some embodiments, treatment with the methods and compositions of the present invention may be applied to infected tissue or contaminated surfaces. In such embodiments, the tissue or surface being treated is in contact with a recipient cell (e.g., a pathogen cell) prior to the exposure of the infected tissue or contaminated surface to the compositions (e.g., donor cells, treated surfaces) of the present invention.

In some embodiments, treatment with the methods and compositions of the present invention may be prophylactic or preventative. In such embodiments, the tissue or surface being treated is not in contact with a recipient cell (e.g., a pathogen cell) prior to the exposure of the tissue or surface to the compositions (e.g., donor cells, treated surfaces) of the present invention. It is contemplated that the methods and compositions of the present may make use of many different recombinant transmissible plasmids. In some embodiments, the recombinant transmissible plasmid is self-transmissible, while in other embodiments, the recombinant transmissible plasmid is not self-transmissible. In some preferred embodiments, the recombinant transmissible plasmid is selected from the group consisting of pCON15-56A, pCON19-79. Helper plasmids include but are not limited to pCON1-93 and pCON1-94.

Recipient cells targeted by the methods and compositions of the present invention include but are not limited to bacterial cells. In preferred embodiments, the recipient cell is a pathogenic bacterial cell. In particularly preferred embodiments, the recipient bacterial cell is of a genus selected from the group consisting of Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, or Chlamydiae.

The present invention provides compositions comprising a donor cell wherein said donor cell comprises a recombinant transmissible plasmid comprising a gene encoding a bactericidal protein and a helper plasmid comprising a gene encoding an immunity protein, wherein said immunity protein is configured to inhibit said bactericidal protein. In such embodiments, the donor cell is configured to conjugatively transfer the recombinant transmissible plasmid to a recipient cell, such that the recombinant transmissible plasmid expresses the gene encoding a bactericidal protein in the recipient cell. In preferred embodiments, expression of the gene encoding a bactericidal protein is lethal to the recipient cell. In some embodiments, the bactericidal protein is a colicin. In some preferred embodiments, the colicin is colE3, while in other preferred embodiments, the bactericidal protein includes but is not limited to colA, colB, colD, colIa, colIb, colK, colN, colE1, colE2, colE4, colE5, colE6, colE7, colE8, colE9, or lysozyme. In some embodiments of the present invention the transmissible plasmid comprises oriT and ori V of RSF 1010, and wherein said gene encoding ColE3 is under control of a lac promoter/operator. In some preferred embodiments, the transmissible plasmid is pCON15-56A or pCON19-79. The compositions of the present invention contemplate the use of an immunity protein configured to inhibit the effects of the bactericidal protein. In preferred embodiments, the immunity protein binds to the bactericidal protein. For example, the immunity protein immE3 binds to and inhibits (e.g., inactivates) the bactericidal protein colE3. Numerous pairs of bactericidal proteins and corresponding immunity proteins are known in the art. In the present invention, the bactericidal proteins listed above are inhibited by the corresponding colicin A, colicin B, colicin D, colicin Ia, colicin Ib, colicin K, colicin N, colicin E1, colicin E2, colicin E4, colicin E5, colicin E6, colicin E7, colicin E8, and colicin E9 immunity proteins, respectively. In some embodiments the gene encoding an immunity protein is under control of a promoter, wherein said promoter is constitutively active. In some embodiments, the promoter is Pneo. In other embodiments, the gene encoding an immunity protein is under control of a promoter that is inducible. In some embodiments, the helper plasmid is pCON1-93 or pCON1-94.

It is contemplated that the compositions of the present invention may be used to treat surfaces. Surfaces that can be treated by the methods and compositions of the present invention include but are not limited to a surfaces of a medical device, a wound care device, a body cavity device, a human body, an animal body, a personal protection device, a birth control device, and a drug delivery device. Surfaces include but are not limited to silicon, plastic, glass, polymer, ceramic, photoresist, skin, tissue, nitrocellulose, hydrogel, paper, polypropylene, cloth, cotton, wool, wood, brick, leather, vinyl, polystyrene, nylon, polyacrylamide, optical fiber, natural fibers, nylon, metal, rubber and composites thereof. In some embodiments, the treating inhibits growth of recipient cells on the surface, while in other embodiments, the treatment kills or attenuates recipient cells that come into contact with the surface.

### DESCRIPTION OF THE DRAWINGS

FIG. 1 depicts methods of monitoring conjugation efficiency. FIG. 1A shows a schematic diagram of an exemplary conjugation assay. FIG. 1B provides an example of a dilution assay to calculate conjugation efficiency.

FIG. 2 shows an image of bacteria spotted on a culture medium for monitoring conjugation and killing efficiency of a killer plasmid.

FIG. 3 show a graph showing the results of an in vivo efficacy test using donor cells containing plasmids of the present invention.

FIG. 4A provides a schematic diagram of an exemplary method for testing bacterial inhibition by donor cells according to the present invention. FIG. 4B shows images of lawns of the indicated target cells (in column ‘a’) and cleared areas in the lawns of pathogen cells from conjugation-dependent growth inhibition (in column ‘b’).

FIG. 5 shows schematic diagrams of plasmids RSF1010 pCON15-56A.

FIG. 6 shows a schematic diagram of plasmid pCON1-94.

FIG. 7 shows a schematic diagram of pCON19-79.

FIG. 8 shows a schematic diagram of pCON1-93.

FIG. 9 shows a graph showing the results of in vivo efficacy testing using donor cells comprising the pCON19-79 plasmid.

### DEFINITIONS

To facilitate an understanding of the present invention, a number of terms and phrases are defined below:

As used herein, the term “subject” refers to individuals (e.g., human, animal, or other organism) to be treated by the methods or compositions of the present invention. Subjects include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of donor cell, and optionally one or more other agents) for a condition characterized by the presence of pathogenic bacteria, or in anticipation of possible exposure to pathogenic bacteria.

The term “diagnosed,” as used herein, refers to the to recognition of a disease (e.g., caused by the presence of pathogenic bacteria) by its signs and symptoms (e.g., resistance to conventional therapies), or genetic analysis, pathological analysis, histological analysis, and the like.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “conjugation” refers to the process of DNA transfer from one cell to another. Although conjugation is observed primarily between bacterial cells, this process takes place from bacterial cells to higher and lower eukaryote cells (Waters, Nat Genet. 29:375-376 (2001); Nishikawa et al., Jpn J Genet. 65:323-334 (1990)). Conjugation is mediated by complex cellular machinery, and essential protein components are often encoded as a series of genes in a plasmid (e.g., the tra genes for plasmid RK2). Some of these gene products are assembled to facilitate a direct cell-cell interaction (e.g., mating pair formation), and some of them serve to transfer DNA and associated protein molecules, and to replicate the DNA molecule (e.g., DNA transfer/replication). oriT is a DNA sequence from which the transfer of a DNA molecule initiates in the process of conjugation.

As used herein, the terms “conjugation donor” and “donor cell” are used interchangeably to refer to a cell, e.g., a bacterial cell, carrying a plasmid, wherein said plasmid can be transferred to another cell through conjugation. Examples of donor cells include, but are not limited to E. coli strains that contain a self-transmissible plasmid or a non-self-transmissible plasmid. A cell receiving a plasmid or other cellular material from a donor cell via conjugative transfer is referred to as a “recipient cell”. As used herein, the term “transmissible plasmid” refers to a plasmid that can be transferred from a donor cell to a recipient cell via conjugation.

As used herein, the term “self-transmissible plasmid” refers to a plasmid encoding all the genes needed to mediate conjugation. A recipient of a self-transmissible plasmid becomes a proficient donor to further transfer the self-transmissible plasmid to another recipient cell.

As used herein, the term “non-self-transmissible plasmid” or “mobilizable plasmid” refers to a plasmid lacking some of the genes needed to mediate conjugation. A cell carrying a non-self-transmissible plasmid does not transfer DNA through conjugation unless the missing gene(s) are supplied in trans within the same cell. Therefore, a recipient cell that lacks the missing gene(s), does not become a proficient conjugation donor when it receives the non-self-transmissible plasmid .

As used herein, the term “origin of transfer” or “orit” refers to the cis-acting site required for DNA transfer, and integration of an oriT sequence into a non-transmissible plasmid converts it into a mobilizable plasmid (Lanka and Wilkins, Annu Rev Biochem, 64:141-169 (1995)).

In some embodiments, a donor cell is a bacterial cell (e.g., a Gram-positive or Gram-negative bacterium). Examples of donor cells include, but are not limited to, bacterial cells of a genus of bacteria, selected from the group comprising Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae.

In some embodiments, a donor cell is a non-viable cell, including but not limited to a bacterial minicell, a maxicell, or a non-dividing cell.

As used herein, the term “maxicell” refers to the cells that have been treated to maximize chromosomal degradation, e.g., by UV irradiation and extended incubation. Maxicells contain mostly plasmid DNA, and synthesis of proteins within maxicells occurs essentially exclusively from the plasmid DNA in the cells.

As used herein, the term “non-dividing cell” or “ND cell” refers to cells that are treated in a manner selected to preferentially damage the chromosomal DNA of the cell (e.g., by UV or other irradiation), wherein said cells are further treated, e.g., by rapid chilling after DNA damaging treatment, to minimize chromosomal degradation. “ND cells” can also be obtained in a process such as temporal expression of bactericidal protein (e.g., ColE3) within a donor bacterium. Thus, in some embodiments, induction of proteins (e.g., ColE3) destroys the protein synthesis in the cell, leading to cell death while leaving the conjugation apparatus and chromosomal DNA synthesized prior to ColE3 synthesis intact. ND cells contain both chromosomal and plasmid DNA but the function of the cell is sufficiently altered, e.g., by UV irradiation, that said ND cells have little or no capability to divide.

The terms “target cells,” “targets,” “recipient cells,” and “recipients” are used interchangeably herein. In preferred embodiments, the target cells for the compositions and methods of the present invention include, but are not limited to, microorganisms such as pathogenic organisms (e.g., pathogenic bacteria) that can receive material from a donor cell via conjugative transfer. Pathogenic bacteria include, but are not limited to, Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae. In some embodiments, target cells are continuously cultured cells. In some embodiments, target cells are uncultured cells existing in their natural environment (e.g., at the site of a wound or infection) or obtained from patient tissues (e.g., via a biopsy). In preferred embodiment, target cells exhibit pathological growth or proliferation.

As used herein, the term “virulence” refers to the degree of pathogenicity of a microorganism, e.g., as indicated by the severity of the disease produced or its ability to invade the tissues of a subject. It is generally measured experimentally by the median lethal dose (LD₅₀) or median infective dose (ID₅₀). The term may also be used to refer to the competence of any infectious agent to produce pathologic effects.

The term “killer gene” refers to a gene that, upon expression in a susceptible cell, produces a product that kills the cell.

The term “killer plasmid” refers to plasmid comprising a killer gene.

As used herein, the terms “attenuate” and “attenuation” as used herein in reference to a feature e.g., of a recipient or target cell, refers to a reducing or weakening of that feature, or a reducing of the effect(s) of that feature. For example, when used in reference to a pathogen or the pathogenicity of a target cell, attenuation generally refers to a reduction in the virulence of the pathogen. Attenuation of a pathogen is not limited to any particular mechanism of reduced virulence. In some embodiments, reduced virulence maybe achieved, e.g., by disruption of a secretory pathway. In other embodiments, reduced virulence may be achieved by altering cellular metabolism to increase reactivity to or susceptibility to a drug, e.g., a drug that attenuates virulence of the pathogen, or that kills the pathogen. In some embodiments, attenuation refers to a feature, e.g., virulence of a population of cells. For example, in some embodiments of the present invention, a population of pathogen cells is treated, e.g., by the methods and compositions of the invention, such that the population of cells is decreased in virulence. See, for example, co-pending application filed May 26, 2005, mailed under Express Mail Label No. EV618124302US, which is incorporated herein by reference in its entirety for all purposes.

As used herein, the term “virulence” refers to the degree of pathogenicity of a microorganism, e.g., as indicated by the severity of the disease produced or its ability to invade the tissues of a subject. It is generally measured experimentally by the median lethal dose (LD₅₀) or median infective dose (ID50). The term may also be used to refer to the competence of any infectious agent to produce pathologic effects.

As used herein, the term “effective amount” refers to the amount of a composition (e.g., donor cells) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., compositions of the present invention) to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

As used herein, the term “treating a surface” refers to the act of exposing a surface to one or more compositions of the present invention. Methods of treating a surface include, but are not limited to, spraying, misting, submerging, and coating.

As used herein, the term “co-administration” refers to the administration of at least two agent(s) (e.g., two separate donor bacteria, each comprising a different plasmid) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., donor bacteria cells) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells which line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like.. The compositions also can include stabilizers and preservatives. For examples of carriers, stabilizers, and adjuvants. (See e.g., Martin, Remington's Pharmaceutical Sciences, 15th Ed., Mack Publ. Co., Easton, Pa. (1975), incorporated herein by reference). Moreover, in certain embodiments, the compositions of the present invention may be formulated for horticultural or agricultural use. Such formulations include dips, sprays, seed dressings, stem injections, sprays, and mists.

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “medical devices” includes any material or device that is used on, in, or through a subject's or patient's body, for example, in the course of medical treatment (e.g., for a disease or injury). Medical devices include, but are not limited to, such items as medical implants, wound care devices, drug delivery devices, and body cavity and personal protection devices. The medical implants include, but are not limited to, urinary catheters, intravascular catheters, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Drug delivery devices include, but are not limited to, needles, drug delivery skin patches, drug delivery mucosal patches and medical sponges. Body cavity and personal protection devices, include, but are not limited to, tampons, sponges, surgical and examination gloves, and toothbrushes. Birth control devices include, but are not limited to, intrauterine devices (IUDs), diaphragms, and condoms.

As used herein, the term “therapeutic agent,” refers to compositions that decrease the infectivity, morbidity, or onset of mortality in a subject contacted by a pathogenic microorganism or that prevent infectivity, morbidity, or onset of mortality in a host contacted by a pathogenic microorganism. As used herein, therapeutic agents encompass agents used prophylactically, e.g., in the absence of a pathogen, in view of possible future exposure to a pathogen. Such agents may additionally comprise pharmaceutically acceptable compounds (e.g., adjutants, excipients, stabilizers, diluents, and the like). In some embodiments, the therapeutic agents of the present invention are administered in the form of topical compositions, injectable compositions, ingestible compositions, and the like. When the route is topical, the form may be, for example, a solution, cream, ointment, salve or spray.

As used herein, the term “pathogen” refers a biological agent that causes a disease state (e.g., infection, cancer, etc.) in a host. “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are Gram-negative or Gram-positive. “Gram-negative” and “Gram-positive” refer to staining patterns with the Gram-staining process, which is well known in the art. (See e.g., Finegold and Martin, Diagnostic Microbiology, 6th Ed., CV Mosby St. Louis, pp. 13-15 (1982)). “Gram-positive bacteria” are bacteria that retain the primary dye used in the Gram stain, causing the stained cells to generally appear dark blue to purple under the microscope. “Gram-negative bacteria” do not retain the primary dye used in the Gram stain, but are stained by the counterstain. Thus, Gram-negative bacteria generally appear red.

As used herein, the term “microorganism” refers to any species or type of microorganism, including but not limited to, bacteria, archaea, fungi, protozoans, mycoplasma, and parasitic organisms. The present invention contemplates that a number of microorganisms encompassed therein will also be pathogenic to a subject.

As used herein, the term “fungi” is used in reference to eukaryotic organisms such as the molds and yeasts, including dimorphic fungi.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1 -methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). A polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences, or 5′ flanking sequences. Sequences located 3′ or downstream of the coding region and present on the mRNA are referred to as 3′ non-translated sequences or 3′ flanking sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into pre-mRNA; introns may contain regulatory elements such as enhancers. Introns are generally removed or “spliced out” from the primary (pre-mRNA) transcript; introns therefore are generally absent in the messenger RNA (mRNA) transcript. The mRNA functions during translation to specify the sequence or order of amino acids in a nascent polypeptide.

As used herein, the term “heterologous gene” and “heterologous nucleic acid” refers to a gene or nucleic acid that is not in its natural environment. For example, a heterologous gene or nucleic acid includes a gene or nucleic acid from one species introduced into another species. A heterologous gene or nucleic acid also includes a gene or nucleic acid native to an organism that has been altered in some way (e.g., mutated, added in multiple copies, linked to non-native regulatory sequences, etc). Heterologous genes or nucleic acids are distinguished from endogenous genes or nucleic acids in that the heterologous gene or nucleic acid sequences are typically joined to DNA sequences that are not found naturally associated with the gene or nucleic acid sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “wild-type” refers to a gene or gene product in the form that would be isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The sequence of nucleotides in the DNA thus encodes for the sequence of amino acids in the corresponding polypeptide.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids” bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The term “homology” refers to a degree of similarity between molecules such as nucleic acid molecules. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i. e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any nucleic acid that can hybridize to either or both strands of the double-stranded nucleic acid sequence under conditions of low stringency as described above. When used in reference to a single-stranded nucleic acid sequence, the term “substantially homologous” refers to any nucleic acid that can hybridize (i.e., it is the complement of) to the complement of the single-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample. In yet another example, nucleic acids in a sample are purified by removing or reducing one or more components from a sample. Components to be reduced or removed in purification comprise other nucleic acids, damaged nucleic acids, proteins, salts, etc.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “transdominant negative mutant gene” refers to a gene encoding a protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

As used herein, the term “kit” refers to any delivery system for delivering materials. In the context of reaction materials such as donor cells, such delivery systems include systems that allow for the storage, transport, or delivery of reaction reagents (e.g., cells, buffers, selection reagents, etc., in the appropriate containers) and/or supporting materials (e.g., media, written instructions for performing using the materials, etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain cells for a particular use, while a second container contains selective media. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction materials needed for a particular use in a single container (e.g., in a single box housing each of the desired components). The term “kit” includes both fragmented and combined kits.

As used herein, the term “cellular metabolic function” refers to any or all, processes conducted by a cell (e.g., enzymatic or chemical processes associated with cell function), other than genomic replication.

### DETAILED DESCRIPTION OF THE INVENTION

Many types of patients being treated for skin lesions require prolonged hospitalization, multiple surgeries, medical interventions and blood transfusions (e.g., bum victims or diabetic patients with chronic ulcers). Several studies indicate a causal relationship between the severity of trauma and surgery and the predisposition of these patients to develop sepsis (see, e.g., Angele and Faist, Crit Care 6, 298 (2002); Roumen et al., Ann Surg 218, 769 (1993)).

Unresolved sepsis leads to multi-organ failure and ultimately death. Organ failure is the leading cause of death in trauma and surgical patients. Excessive inflammatory response and depression of cell-mediated immunity predisposes these patients to infectious complications (see, e.g., Angele and Faist, Crit Care 6, 298 (2002); Faist, Curr Top Microbiol Immunol 216, 259 (1996); and Schinkel et al., J Trauma 44, 743 (1998)).

In recent years, the emergence of several multi-antibiotic-resistant bacterial strains has made the treatment of nosocomial infections in critically injured patients exceedingly difficult. Methicillin resistant Staphylococcus aureus and multi-drug-resistant strains of Pseudomonas aeruginosa and Acinetobacter baumannii (A. baumannii) are among some of most difficult infections to control and eradicate in critically injured patients. A. baumannii is often either pan-drug resistant or susceptible only to the extremely toxic antibiotic Colistin. Outbreaks of A. baumannii are becoming more common and widespread. Novel strategies are needed that will provide an efficient and potent therapeutic arsenal to fight these pan-resistant infections.

Thus, in some embodiments, the present invention provides a therapeutic treatment comprising donor cells (e.g., pathogenic or non-pathogenic bacteria, non-dividing cells) comprising one or more plasmids (e.g., self-transmissible or non-self-transmissible plasmids), wherein the plasmid may be transferred (e.g., through conjugation) from the donor cell to a target/recipient cell (e.g., a pathogenic microorganism), resulting in the plasmid expressing its genetic material in the target.

In some embodiments, the present invention provides donor cells comprising a transmissible plasmid, wherein the plasmid may be transferred (e.g., through conjugation) from the donor cell to a recipient cell (e.g., a pathogenic microorganism), resulting in the plasmid expressing its genetic material in the recipient cell, so as to alter a cellular function, e.g., a virulence factor, of the recipient cell. In preferred embodiments, the transmissible plasmid is a recombinant transmissible plasmid. Conjugation for transferring genetic material from a donor cell into a target recipient cell for a variety of purposes has been described. See, e.g., PCT Publication WO 02/18605, U.S. Patent Application Ser. No. 20040137002, and U.S. patent application Ser. No. 10/884,257, each incorporated herein by reference in its entirety for all purposes. The present invention makes use of conjugative transfer to alter the cellular functions of a recipient cell, e.g., to kill or impair the target cell.

It is contemplated that alteration of recipient cells according to the present invention also comprises altering such cells so as to alter the response of such recipient cells to drugs, e.g., antibiotics. It is contemplated that any alteration to a recipient cell to alter that recipient cell's metabolism such that said recipient cell becomes susceptible to, or has increased susceptibility or response to a drug is encompassed by the methods, compositions and systems of the present invention. In some embodiments, a transmissible plasmid of the present invention encodes a factor capable of inhibiting a pathogen's ability to destroy or inactivate a drug such as an antibiotic. For example, an expression product of a transmitted plasmid may disrupt the ability of a pathogen enzyme capable of destroying or inactivating an antibiotic. In other embodiments, an expression product of a transmitted plasmid may provide a receptor for an antibiotic on or in the pathogen cell, or restore a defective receptor for an antibiotic on or in the pathogen cell. In yet other embodiments, an expression product of a transmitted plasmid may facilitate entry of an antibiotic into the pathogen cell, or inhibit the pathogen cell's ability to transport the antibiotic out of the pathogen cell.

In some embodiments, an expression product of a transmitted plasmid may serve to metabolize an inactive drug such as a prodrug into an active form, e.g., a form to which the recipient cell is responsive. The use of prodrugs that are metabolized to form an active drug can be particularly beneficial in bypassing drug resistance mechanisms, and in providing selective treatment, e.g., targeting cells that have received an appropriate transmissible plasmid.

Transmissible Plasmids

The RK2 conjugation system is a very proficient process of DNA transfer from Gram-negative bacterial hosts (e.g., E. coli), and the RK2 plasmid can even conjugate through kingdoms (see, e.g., Bates et al., J Bacteriol 180, 6538-6543 (1998); Waters, Nat Genet 29, 375-376 (December 2001)). RK2 is not capable of stably replicating in animal or yeast cells, but DNA transfer takes place. Thus, the functional RK2 conjugation machinery can mobilize a plasmid DNA from a large number of Gram-negative bacterial hosts. It has been shown that, as long as proper vegetative replication origins are introduced, a plasmid can be mobilized from these donors (E. coli and other Gram-negative donors) to other Gram-negative target strains, and even Gram-positive target strain (see, e.g., Giebelhaus et al., J Bacteriol 178, 6378-6381 (1996)), generating exconjugants. Conjugation systems of the present invention are not limited to RK2, since the majority of conjugative plasmids share strong similarities, and any other system could serve as a delivery system.

For example, it is contemplated that multiple other conjugative systems are suitable for use in the present invention, including, but not limited to RK2, R6K, pCU1, p15A, pIP501, pAMI, pCRG1600. In some embodiments, two or more conjugation systems are used concurrently. In addition to those already described, exemplary plasmids that find use in the present invention include, but are not limited to, those of U.S. Pat. App. Nos. 20040137002, 20040224340, and 10/884,257, herein incorporated by reference in their entireties for all purposes.

Killer Plasmids

While an understanding of the mechanism is not necessary to practice the present invention and while the present invention is not limited to any particular mechanism of action, it is contemplated that, in some embodiments, donor cells comprise a transmissible plasmid that is conjugatively transferred into a target, wherein one or more products encoded by the plasmid are expressed (e.g., to make mRNA or protein) resulting in the killing of the target cells or the inhibiting of their growth (see, e.g., Examples 6 and 7). In some embodiments, the donor cells further comprise a helper plasmid. In some embodiments, the transmissible plasmid is a self-transmissible plasmid.

In some embodiments, donor cells comprise a non-self-transmissible plasmid (e.g., pCON 15-56A) comprising nucleic acid that encodes a polyamino acid (e.g., a polypeptide or a protein) that is bactericidal. In preferred embodiments, donor cells further comprise nucleic acid that encodes a polyamino acid capable of neutralizing the bactericidal properties of the polyamino acid of the non-self-transmissible plasmid within the donor cells (e.g., an immunity protein; see, e.g., Examples 2 and 4). In preferred embodiments, the gene encoding the neutralizing polyamino acid is on a helper plasmid, including but not limited to pCON1-93 or pCON1-94. In some embodiments, the polyamino acid capable of neutralizing the bactericidal polyamino acid is under control of a constitutive promoter. In some embodiments, the polyamino acid capable of neutralizing the bactericidal polyamino acid is under control of an inducible promoter. While an understanding of the mechanism is not necessary to practice the present invention and while the present invention is not limited to any particular mechanism of action, it is contemplated that, in some embodiments, the polyamino acid capable of neutralizing bactericidal polyamino acid and the bactericidal polyamino acid form a non-toxic complex within the donor bacteria, the complex is secreted outside of the donor bacteria, the complex or component parts bind to receptors on the target cells, are translocated into the target cells and target cell death ensues.

In some embodiments, the bactericidal polyamino acid is encoded by the colE3 gene. The present invention is not limited by the type of bactericidal gene used. Indeed a variety of bactericidal genes are contemplated including, but not limited to, colA, colB, colD, colIa, colIb, colK, colN, colE1, colE2, colE4, colE5, colE6, colE7, colE8, colE9 and a gene encoding lysozyme. In some embodiments, the self-transmissible or non-self-transmissible plasmid comprises a promoter (e.g., the lac promoter/operator) that drives expression of the bactericidal polyamino acid. In some embodiments, the helper plasmid encodes a repressor protein (e.g., lacd) capable of inhibiting expression of the bactericidal gene. In some embodiments, the repressor protein is under control of a constitutive promoter. In some embodiments, the repressor protein is under control of an inducible promoter.

As described above, in preferred embodiment, the donor cells of the present invention comprise an immunity protein that inhibits or neutralizes the bactericidal protein expressed by the transmissible plasmid. Numerous pairs of bactericidal proteins and corresponding immunity proteins are known in the art. In the present invention, the bactericidal proteins listed above are inhibited by the corresponding colicin A, colicin B, colicin D, colicin Ia, colicin Ib, colicin K, colicin N, colicin E1, colicin E2, colicin E4, colicin E5, colicin E6, colicin E7, colicin E8, and colicin E9 immunity proteins, respectively. Still other combinations of bactericidal proteins (e.g., bacteriocins) and neutralizing immunity proteins are known in the art (see, e.g., exemplary tables of bacteriocin immunity proteins on the World Wide Web site us.expasy.org/cgi-bin/get-entries?KW=Bacteriocin%20immunity, the ExPASy (Expert Protein Analysis System) proteomics server of the Swiss Institute of Bioinformatics (SIB)). In some embodiments the gene encoding an immunity protein is under control of a promoter, wherein said promoter is constitutively active. In some embodiments, the promoter is Pneo. In other embodiments, the gene encoding an immunity protein is under control of a promoter that is inducible. In some embodiments, the helper plasmid is pCON1-93 or pCON1-94.

A variety of self-transmissible and non-self-transmissible plasmids are contemplated in the present invention. For example, in some embodiments, the present invention utilizes the plasmid RSF1010 as a backbone for construction of plasmids. In some embodiments, the plasmids are derivatives of pACYC177. It is contemplated that the compositions comprising plasmids of the present invention find use in research and therapeutic applications.

Donor Cells

Donor Bacteria

It is contemplated that any type of bacteria (e.g., Gram-positive and Gram-negative bacteria) can be used as donor cells in the present invention (see, e.g., Example 1). A number of approaches may be taken to prevent spread (e.g., growth) of donor bacteria. In addition to using non-dividing cells as donors (see, e.g., U.S. patent application Ser. No. 10/884,257, filed Jul. 2, 2004, herein incorporated by reference in its entirety for all purposes), several other approaches include, but are not limited to, using donors with temperature-sensitive mutations (e.g., aminoacyl-tRNA synthetase and RNase P mutations), auxotrophic mutants (e.g., dapA and aroA), serine mutations, and/or other mutations or deficiencies in amino acid synthesis. These examples are not meant to limit the scope of the invention. Those skilled in the art will immediately appreciate that there are alternative approaches that may be used to attenuate donor bacteria. These mutations have been analyzed and are known well in the art, and introduction of these mutations into a newly obtained bacterial donor is well within the capabilities of one of skill in the art.

In some embodiments, donor bacterial cells of the present invention comprise temperature sensitive mutation(s). A temperature-sensitive mutant grows abnormally within a certain range of temperature compared to its isogenic wild-type bacteria. In the mutant, a mutation in the RNA or protein causes effects, e.g., changes in conformation, that are sensitive to temperature such that mutants can be grown in a lab at their permissive temperature; however, they have severe growth defects at non-permissive (e.g., higher) temperatures (e.g., at body temperature).

Examples of these mutations include aminoacyl-tRNA synthetases (see, e.g., Sakamoto et al., J Bacteriol 186, 5899-5905 (2004); Martin et al., J Bacteriol 179, 3691-3696 (1997)), and RNase P (Li, Rna 9, 518-532 (2003); Li and Altman, Proc Natl Acad Sci U S A 100, 13213-13218 (2003)). An aminoacyl-tRNA synthetase catalyzes the esterification of a specific amino acid to the 3′-terminal adenosine of the corresponding tRNA, and RNase P is an crucial ribonuclease to generate the mature 5′ end of tRNAs in all organisms (Gopalan et al., J Biol Chem 277, 6759-6762 (2002). Defects in these enzymatic functions prevent protein synthesis in the cell.

In some embodiments (e.g., when Gram-positive bacteria are targets) Gram-positive donors are used. Gram-positive donor bacteria include, but are not limited to, Bacillus sp., Staphylococcus sp., Enterococcus sp., Streptococcus sp., Lactobacillus sp. and Lactococcus sp. Of these strains, Lactobacillus and Lactococcus are particularly useful because these species have been used in food industry, and categorized as GRAS (Generally Recognized As Safe) in Title 21 of the Code of Federal Regulations (CFR). For these Gram-positive hosts, it is possible to use, among other plasmids, the conjugative plasmids pAD1 and/or pCF10, two of the best-studied Gram-positive conjugative plasmids (see, e.g., Hirt et al., J Bacteriol 187, 1044-1054 (2005); Francia et al., Plasmid 46, 117-127 (2001)). Conjugation machineries of these plasmids share significant levels of similarity with RK2. Based on the literature, it is contemplated that these plasmids can be modified (see, e.g., Example 2) for use in the present invention. These modifications include, among other things, generation of mobilizable plasmids, integration of bactericidal genes, and addition and subtraction of restriction enzyme cut sites.

In preferred embodiments of the present invention, certain features are employed in the plasmids and donor cells of the invention to minimize potential risks associated with the use of DNA or genetically modified organisms in the environment. For instance, in environmentally sensitive circumstances it may be preferable to utilize non-self-transmissible plasmids. Thus, in some embodiments, the plasmids are mobilizable by conjugative machinery but are not self-transmissible. As discussed herein, this may be accomplished in some embodiments by integrating into the host chromosome all tra genes whose products are necessary for the assembly of conjugative machinery. In such embodiments, plasmids are configured to possess only an origin of transfer (oriT). This feature prevents the recipient, before or even after it dies, from transferring the plasmid further.

Another biosafety feature comprises utilizing conjugation systems with predetermined host-ranges. As discussed above, certain elements are known to function only in few related bacteria (narrow-host-range) and others are known to function in many unrelated bacteria (broad-host-range or promiscuous) (del Solar et al., Mol. Microbiol. 32: 661-666, (1996); Zatyka and Thomas, FEMS Microbiol. Rev. 21: 29 1 319, (1998)). Also, many of those conjugation systems can function in either Gram-positive or Gram-negative bacteria but generally not in both (del Solar, 1996, supra; Zatyka and Thomas, 1998, supra).

In some embodiments, donor bacterial cells of the present invention comprise auxotrophic mutant(s). There are large numbers of auxotrophic mutants known in the art. Examples of genes causing such phenotype are dapA and aroA. dapA encodes an enzyme dihydropicolinate synthase, a key enzyme for lysine biosynthesis in plant and bacteria (see, e.g., Ledwidge and Blanchard, Biochemistry 38, 3019-3024 (1999)), and aroA encodes an enzyme 5-enolpyruvylshikimate 3-phosphate synthase, catalyzing a key step in the synthesis of aromatic amino acids (see, e.g., Rogers et al., Appl Environ Microbiol 46, 37-43 (1983)).. These mutants can be grown under laboratory conditions with the supplement of lacking amino acids for these bacteria. However, upon application, these mutants cannot grow well because the key nutritional factor is missing. These are but two examples, and there are many similar auxotrophic mutations known to be available to those of skill in the art.

Inadvertent proliferation of antibiotic resistance is minimized in this invention by avoiding the use of antibiotic resistance markers. In a preferred alternative approach, the gene responsible for the synthesis of an amino acid (e.g. serine) can be mutated, generating the requirement for this amino acid in the donor. Such mutant bacteria will prosper on media lacking serine provided that they contain a plasmid with the ser gene whose product is needed for growth.

Thus, the invention contemplates the advantageous use of plasmids containing the Ser gene or one of many other nutritional genetic markers. These markers permit selection and maintenance of the plasmids in donor cells.

Another approach comprises the use of restriction-modification systems to modulate the host range of plasmids. Conjugation and plasmid establishment are expected to occur more frequently between taxonomically related species in which plasmid can evade restriction systems and replicate. Type II restriction endonucleases make a double-strand break within or near a specific recognition sequence of duplex DNA. Cognate modification enzymes can methylate the same sequence and protect it from cleavage. Restriction-modification systems (RM) are ubiquitous in bacteria and archaebacteria but are absent in eukaryotes. Some of RM systems are plasmid-encoded, while others are on the bacterial chromosome (Roberts and Macelis, Nucl. Acids Res. 24: 223-235, (1998)). Restriction enzymes cleave foreign DNA such as viral or plasmid DNA when this DNA has not been modified by the appropriate modification enzyme. In this way, cells are protected from invasion of foreign DNA. Thus, by using a donor strain producing one or more methylases, cleavage by one or more restriction enzymes could be evaded. Site directed mutagenesis is used to produce plasmid DNA that is either devoid of specific restriction sites or that comprises new sites, protecting or making plasmid DNA vulnerable, respectively against endonucleases. In some embodiments, broad-host range plasmids are used that evade restriction systems simply by not having many of the restriction cleavage sites that are typically present on narrow-host plasmids (Wilkins et al., J. Mol. Biol 258, 447-456 (1996)).

In some embodiments, the present invention utilizes environmentally safe bacteria as donors. Safe bacteria are known in the art. For example, delivery of DNA vaccines by attenuated intracellular Gram-positive and Gram-negative bacteria has been reported (Dietrich et al., 2001 Vaccine 19, 2506-2512; Grillot-Courvalin et al., 1999 Current Opinion in Biotech. 10, 477-481). In addition, the donor strain can be one of thousands of harmless bacteria that colonize the non-sterile parts of the body (e.g., skin, gastrointestinal, urogenital, mouth, nasal passages, throat and upper airway systems).

Non-Viable Donor Cells

In another strategy non-viable donors are utilized instead of living cells. For example, minicells and maxicells are well studied model systems of metabolically active but nonviable bacterial cells. Minicells lack chromosomal DNA and are generated by special mutant cells that undergo cell division without DNA replication. If the cell contains a multicopy plasmid, many of the minicells will contain plasmids. Minicells neither divide nor grow. However, minicells that possess conjugative plasmids are capable of conjugal replication and transfer of plasmid DNA to living recipient cells. (see, e.g., U.S. Pat. No. 4,968,619).

Maxicells are cells that are treated so as to destroy their chromosomal DNA, while retaining the function of plasmids that they contain. Maxicells can be obtained from a strain of E. coli that carries mutations in the key DNA repair pathways (recA, uvrA and phr). Because maxicells lack so many DNA repair functions, they die upon exposure to low doses of UV. Importantly, plasmid molecules (e.g., pBR322) that do not receive UV irradiation continue to replicate. Transcription and translation (plasmid-directed) can occur efficiently under such conditions (Sancar et al., J. Bacteriol. 137: 692-693 (1979)), and the proteins made prior to irradiation should be sufficient to sustain conjugation. This is supported by the following two observations: i) that streptomycin-killed cells remain active donors, and ii) that transfer of conjugative plasmids can occur in the presence of antibiotics that prevent de novo gene expression (see, e.g., Heineman and Ankenbauer, J. Bacteriol. 175. 583-588 (1993); Cooper and Heineman, Plasmid 43, 171-175 (2000)). Accordingly, UV-treated maxicells will be able to transfer plasmid DNA to live recipients. It should also be noted that the conservation of recA and uvrA genes among bacteria should allow maxicells of donor strains other than E. coli to be obtained.

In some embodiments, the present invention utilizes non-dividing cells (e.g., a described in U.S. patent application Ser. No. 10/884,257, filed Jul. 2, 2004, incorporated herein by reference in its entirety for all purposes) as donor cells. Non-dividing cells are generally treated such that the ability to divide and grow is removed but conjugation efficiency is preserved. In preferred embodiments, non-dividing cells are treated such that chromosomal DNA is damaged but is not destroyed to the same extent as it is in the creation of maxicells.

In some embodiments, modified microorganisms that cannot function because they contain temperature-sensitive mutation(s) in genes that encode for essential cellular functions (e.g., cell wall, protein synthesis, RNA synthesis, as described, for example, in U.S. Pat. No. 4,968,619) are used.

For many approaches, conditionally replicating plasmids can be used. Such plasmids, can replicate in the donor but cannot replicate in the recipient bacterium simply because their cognate replication initiator protein (e.g., Rep) is produced in the former cells but not the latter cells. In some embodiments, a variant plasmid contains a temperature-sensitive mutation in the rep gene, so it can replicate only at temperatures below 37C. Hence, its replication will occur in bacteria applied on skin but it will not occur if such bacteria break into the body's core.

In some embodiments, the present invention provides compositions and methods capable of killing any bacterial cell. The present invention is not limited by the type of cells targeted. For example, target bacterial cells include, but are not limited to, those selected from the group consisting of Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae.

In some embodiments, nucleic acid sequences encoding proteins (e.g., bactericidal proteins) are encoded on a transmissible or non-transmissible plasmid (e.g., RK2, R6K, pCU1, p15A, pIP501, pAM1, pCRG1600 or PCON4-78) and placed into a donor cell (e.g., a pathogenic or non-pathogenic genus of bacteria) that posses the ability to conjugatively transfer the plasmid to a recipient cell (e.g., a pathogenic or non-pathogenic genus of bacteria) for expression of the protein. In preferred embodiments, expression of the nucleic acid sequence encoded on the conjugatively transferred plasmids leads to killing of the recipient/target cells. In addition to those described herein, exemplary donor cells that find use in the present invention include, but are not limited to, those of U.S. Pat. App. Nos. 20040137002, 20040224340, and 10/884,257, herein incorporated by reference in their entireties for all purposes.

Therapeutics

The compositions and methods of the present invention find utility for treatment of humans and in a variety of veterinary, agronomic, horticultural and food processing applications.

For human and veterinary use, and depending on the cell population or tissue targeted for protection (e.g., via killing of pathogenic target cells), the following modes of administration of the compositions (e.g., donor bacterial cells comprising a transmissible plasmid) of the present invention are contemplated: topical, oral, nasal, pulmonary/bronchial (e.g., via an inhaler), ophthalmic, rectal, urogenital, subcutaneous, intraperitoneal and intravenous. The bacteria preferably are supplied as a pharmaceutical preparation, in a delivery vehicle suitable for the mode of administration selected for the patient being treated.

For instance, to deliver the donor bacterial cells to the gastrointestinal tract or to the nasal passages, the preferred mode of administration is by oral ingestion or nasal aerosol, or by feeding (alone or incorporated into the subject's feed or food). In this regard, it should be noted that probiotic bacteria, such as Lactobacillus acidophilus, are sold as gel capsules containing a lyophilized mixture of bacterial cells and a solid support such as mannitol. When the gel capsule is ingested with liquid, the lyophilized cells are re-hydrated and become viable, colonogenic bacteria. Thus, in a similar fashion, donor bacterial cells of the present invention can be supplied as a powdered, lyophilized preparation in a gel capsule, or in bulk for sprinkling into food or beverages. The re-hydrated, viable bacterial cells will then populate and/or colonize sites throughout the upper and lower gastrointestinal system, and thereafter come into contact with the target pathogenic bacteria.

For topical applications, the bacteria may be formulated as an ointment or cream to be spread on the affected skin surface. Ointment or cream formulations are also suitable for rectal or vaginal delivery, along with other standard formulations, such as suppositories. The appropriate formulations for topical, vaginal or rectal administration are well known to medicinal chemists.

The present invention will be of particular utility for topical or mucosal administrations to treat a variety of bacterial infections or bacterially related undesirable conditions. Some representative examples of these uses include, but are not limited to, treatment of (1) conjunctivitis, caused by Haemophilus sp., and corneal ulcers, caused by Pseudomonas aeruginosa; (2) otitis externa, caused by Pseudomonas aeruginosa; (3) chronic sinusitis, caused by many Gram-positive cocci and Gram-negative rods, and for general decontamination of bronchii; (4) cystic fibrosis, associated with Pseudomonas aeruginosa; (5) enteritis, caused by Helicobacter pylori (ulcers), Escherichia coli, Salmonella typhimurium, Campylobacter and Shigella sp.; (6) open WO 02/18605 PCT/USOI/27028 associated with Gardnerella vaginalis and other anaerobes; and (12) gingivitis and/or tooth decay caused by various organisms.

The donor cells of the present invention can be applied to skin (e.g., burned or infected skin) as a therapeutic or applied as a prophylactic to prevent bacterial infection. It is contemplated that the donor cells can be applied to the skin surface via a number of delivery mechanisms.

For example, the compositions (e.g., donor cells comprising killer plasmids) of the present invention can be applied (e.g., to a skin burn or wound surface) by multiple methods, including, but not limited to: being suspended in a solution (e.g., colloidal solution) and applied to a surface; being suspended in a solution and sprayed onto a surface using a spray applicator; being mixed with fibrin glue and applied (e.g., sprayed) onto a surface (e.g., skin burn or wound); being impregnated onto a wound dressing or bandage and applying the bandage to a surface (e.g., an infection or wound); being applied by a controlled-release mechanism; being impregnated on one or both sides of an acellular biological matrix that can then be placed on a surface (e.g., skin wound or bum) thereby protecting at both the wound and graft interfaces; being applied as a liposome; or being applied on a polymer.

While an understanding of the mechanism is not necessary to practice the present invention and while the present invention is not limited to any particular mechanism of action, it is contemplated that, in some embodiments, once on the skin or wound surface, donor bacteria come into contact with the targeted pathogenic bacteria and pass antibacterial genes via the conjugation process into the targeted pathogens, killing the pathogens.

Donor bacteria can be any strain of bacteria including any Gram-negative or Gram-positive bacterium. For example, in some embodiments, the present invention provides E. coli, Pseudomonas sp., Klebsiella sp., Enterobacter sp., Acinetobacter sp., Lactobacillus sp., Lactococcus sp., Staphylococcus sp., Streptococcus sp., Enterococcus sp., or Bacteroides sp. as donor bacteria.

In other embodiments, the compositions and methods of the present invention find application in the treatment of surfaces for the attenuation or growth inhibition of unwanted bacteria (e.g., pathogens). For example, surfaces that may be used in invasive treatments such as surgery, catheterization and the like may be treated to prevent infection of a subject by bacterial contaminants on the surface. It is contemplated that the methods and compositions of the present invention may be used to treat numerous surfaces, objects, materials and the like (e.g., medical or first aid equipment, nursery and kitchen equipment and surfaces) to control bacterial contamination thereon.

In other embodiments, the compositions may be impregnated into absorptive materials, such as sutures, bandages, and gauze, or coated onto the surface of solid phase materials, such as surgical staples, zippers and catheters to deliver the compositions to a site for the prevention of microbial infection. Other delivery systems of this type will be readily apparent to those skilled in the art.

Pharmaceutical preparations comprising the donor bacteria are formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of the donor bacteria cells calculated to produce the desired antibacterial (e.g., attenuation of pathogenicity) effect in association with the selected pharmaceutical carrier. Procedures for determining the appropriate dosage unit are well known to those skilled in the art.

Dosage units may be proportionately increased or decreased based on the weight of the patient. Appropriate concentrations for achieving eradication of pathogenic bacteria in a target cell population or tissue may be determined by dosage concentration curve calculations, as known in the art.

Other uses for the donor cells of the invention are also contemplated. These include a variety of agricultural, horticultural, environmental and food processing applications. For example, in agriculture and horticulture, various plant pathogenic bacteria may be targeted in order to minimize plant disease. One example of a plant pathogen suitable for targeting is Erwinia amylovora, the causal agent of fire blight.

Similar strategies may be utilized to reduce or prevent wilting of cut flowers. In veterinary or animal agriculture, the compositions (e.g., plasmid systems) of the invention may be incorporated into animal feed (chicken, cattle) to reduce bio-burden or to attenuate certain pathogenic organisms (e.g., Salmonella). In other embodiments, the invention may be utilized on meat or other foods to attenuate or neutralize pathogenic bacteria (e.g., E. coli 01 57:H7 on meat).

Environmental utilities comprise, for example, engineering Bacillus thuringiensis and one of its conjugative plasmids to deliver and conditionally express insecticidal agents (e.g., for the control of mosquitoes that disseminate malaria or West Nile virus). In such applications, as well as in the agricultural and horticultural applications described above, formulation of the plasmids and donor bacteria as solutions, aerosols, or gel capsules are contemplated.

### EXPERIMENTAL

The following examples are provided in order to demonstrate and further illustrate certain preferred embodiments and aspects of the present invention and are not to be construed as limiting the scope thereof.

In the experimental disclosure that follows, the following abbreviations apply: ° C. (degrees Centigrade); cm (centimeters); g (grams); 1 or L (liters); μg (micrograms); μl (microliters); μm (micrometers); μM (micromolar); μmol (micromoles); mg (milligrams); ml (milliliters); mm (millimeters); mM (millimolar); mmol (millimoles); M (molar); mol (moles); ng (nanograms); nm (nanometers); nmol (nanomoles); N (normal); pmol (picomoles); bp (base pairs); cfu (colony forming units); Invitrogen (Invitrogen, Carlsbad, Calif.); lacOP (region encoding the E. coli lac operator/promoter); Kan (determinant for kanamycin resistance); Cm (determinant for chloramphenicol resistance); Tral (region encoding genes responsible for conjugative transfer); Control (region encoding control region); oriV (region encoding the origin of vegetative replication); oriT (region encoding the origin of conjugative transfer); tetR (gene encoding repressor of tetA); tetA (gene encoding resistance to tetracycline); Rep (region encoding genes responsible for replication); Primase (region encoding genes involved in replication); Tra2 (region encoding genes responsible for mating pair formation); colE3 (gene encoding colicin E3); repA, repB and repC (encode proteins essential for vegetative replication of RSF1010); mobA, mobB and mobC (encodes proteins responsible for mobilization of RSF1010); region encoding iterons, ssiA and ssiB (origins of vegetative replication).

### Example 1

### Donor Used for in vitro and in vivo Testing

Through conjugation, a plasmid can be mobilized in either self-transmissible or non self-transmissible manner. To initiate conjugal transfer products, the tra genes and oriT (origin of transfer) DNA sequence are required. The tra gene products recognize the oriT sequence and initiate nicking one strand within the sequence, and mobilize this single-stranded plasmid DNA into a recipient cell. When all the essential tra genes and the oriT sequence are located on a single plasmid, this plasmid is called self-transmissible since the recipient bacterium of this plasmid becomes a proficient conjugation donor. In contrast, non self-transmissible plasmid carries the oriT sequence, and does not have the entire set of the tra genes. This plasmid can mobilize into a recipient cell only when the tra gene products are supplied in trans in the same donor cell, either from the genes encoded on the chromosome or on the other plasmid. A derivative of E. coli (e.g., K12, S17, see, e.g., Simon et al., Bio/Technology 1, 784-791 (1985)) was used during development of the present invention. These strains have an integrated RK2 plasmid providing all the tra gene products essential for replication and conjugal transfer of mobilizable plasmids such as mini RK2 or IncQ plasmid (e.g. RSF1010). Thus, in some embodiments, the present invention uses self-transmissible plasmids.

In additional to the integrated tra genes, S17-1 is also recA defective, preventing most of homologous recombination in the cell. recA minus E. coli grows significantly slower than its parental strain, and its poor growth is one important factor to prevent the spread of this donor. Further modifications of this strain, for use in compositions and methods of the present invention, are described below.

Lipopolysaccharide (LPS) is one of the major components to trigger inflammation in an animal host upon infection, and S17-1 also carried LPS. LPS is an essential component for bacterial survival; therefore elimination of this molecule is not a plausible approach. However, certain modifications to LPS allow cell growth but significantly reduce the inflammatory response. For example, the msbB gene encodes an enzyme responsible for attaching a myristoyl group to LPS. Elimination of this acyl group from LPS results in a 10 to 100 fold reduction of inflammatory response (see, e.g., Low et al., Nat Biotechnol 17, 37-41 (1999)). Thus, in some embodiments, the present invention provides S17-1 with a deleted (e.g., through gene replacement) msbB gene. We deleted msbB in S17-1 using a common molecular genetic technique, gene replacement (see, e.g., Court et al., Annu Rev Genet 36, 361-388 (2002); and Gong et al., Genome Res 12, 1992-1998 (2002)) The newly generated E. coli donor strain, designed according to this method, was designated as CON4-11c.

The conjugation efficiency of this msbB-defective strain was examined, and no detectable difference was observed in its conjugation efficiency as compared to controls. Therefore, this is a very useful strain for therapeutic applications because it triggers less inflammation without harming the conjugation efficiency. This strain was used in both in vitro and in vivo experiments to demonstrate the usefulness and broad range of application of the compositions and methods of the present invention.

### Example 2

### Construction of Non Self-Transmissible Killer Plasmids

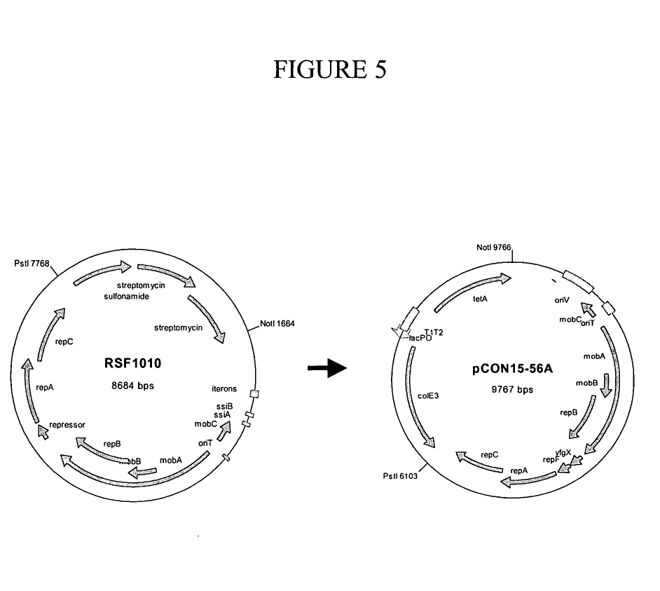

RK2 is a broad-host range plasmid, and able to replicate in almost all Gram-negative bacteria. However, its conjugation efficiency varies depending on different recipient strains, and Pseudomonas aeruginosa is one of these relatively poor conjugation recipients. In contrast, plasmids of the IncQ group (e.g. RSF1010) are mobilizable plasmids, and utilize the tra gene products supplied by RK2 (see, e.g., Lessl et al., J Bacteriol 174, 2493-2500 (1992); Tietze, Microbiol Mol Biol Rev 65, 481-496 (2001)). The conjugation efficiencies of RSF1010 and RK2 were compared using P. aeruginosa as a recipient. The results showed that RSF1010 conjugated approximately 100 times better than RK2 with this bacterium. Accordingly, RSF1010 was used as a backbone for construction of killer plasmids. An example of one such plasmid generated is pCON15-56A (see, e.g., FIG. 5).

In order to generate pCON15-56A, the PstI-NotI fragment of RSF1010 was replaced with PstIl-NotIl fragment carrying tetA from RK2 and colE3 to generate pCON15-56A. colE3 was under the control of the lac promoter/operator, lacPO, which is tightly repressed in the presence of the lac repressor LacI and glucose in the culture medium. In front of lacPO, transcriptional terminators were cloned to prevent leaky expression of colE3 by read-through transcription initiated in front of lacPO. RSF1010 also carries streptomycin and sulfonamide resistant determinants, but they were eliminated in the process of constructing pCON15-56A. A diagram of the vectors is shown below.

In some embodiments, colE3was used as a bactericidal gene. colE3 is tightly repressed on the plasmid as long as glucose is added in the culture medium (see, e.g., Anthony, J Microbiol Methods 58, 243-250 (2004)). This highly potent toxin is a ribonuclease that specifically cleaves a conserved nucleotide sequence at the 3′ end of 16S ribosomal RNA (see, e.g., Bowman et al., Proc Natl Acad Sci U S A 68, 964-8 (1971)). However, leaky expression is observed when the donor carrying this plasmid is exposed to an environment without a high amount of glucose (e.g., at the site of wound). When this happens (i.e., as soon as the bacteria start expressing colE3), the donor cells are killed because of expression of this toxin. To avoid this, a helper plasmid was introduced into the same host bacterium. This helper plasmid, pCONl-94, carries immE3 that encodes an immunity protein for the toxin (see, e.g., Jakes and Zinder, Proc Natl Acad Sci U S A 71, 3380-3384 (1974), and the repressor of lacPO, lacI.

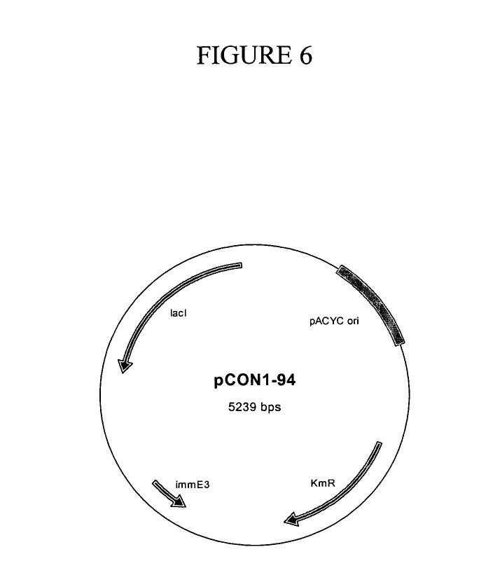

The backbone of the pCON1-94 plasmid is derived from pACYC177. immE3 is expressed using a constitutive promoter Pneo (promoter to express a neomycin-resistance determinant derived from Tn5). lacl is expressed under its own promoter derived from lacI^{Q}. This plasmid has a kanamycin-resistance determinant, KmR. The plasmids, pCON15-56A and pCON1-94, are compatible, and are stably maintained in an E. coli host in the presence of appropriate selective pressures, kanamycin and tetracycline. The structure of pCON1-94 is depicted in FIG. 6.

### Example 3

### Monitoring Conjugation

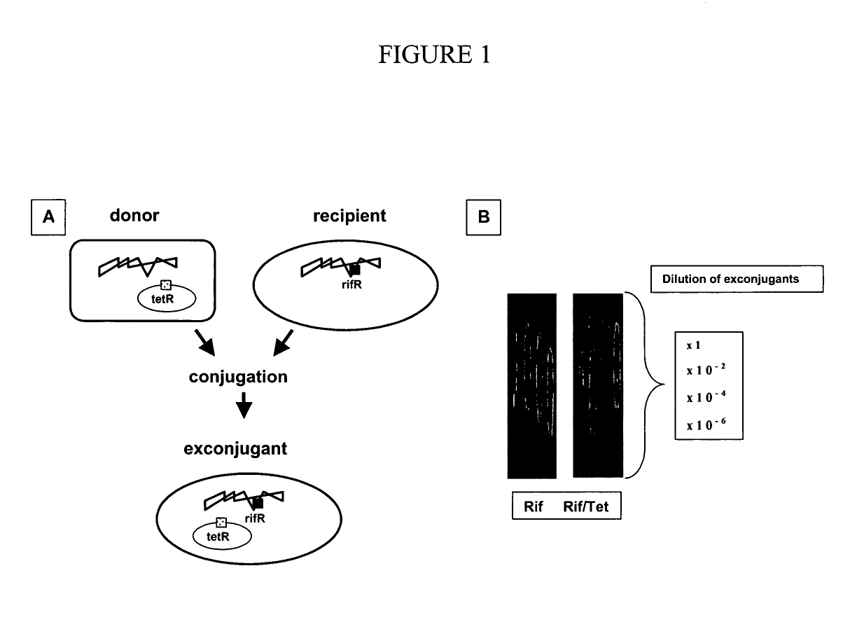

A regular filter conjugation was used to monitor the efficiency of conjugation. This method is well established in the art (Merryweather et al., J Bacteriol 167, 12-17 (1986). The process is depicted in the FIG. 1. After counting the colonies on both plates, efficiency of conjugation was calculated using the equation:

$$\frac{Number of colonies on Rif / Tet per unit volume}{Number of colonies on Rif per unit volume} ⨯ 100 = Conjugation efficiency ( \% )$$

Briefly, donor and target cells were grown overnight in Luria Bertani (LB) medium containing appropriate antibiotics, with the same amount of donor and recipient/target cells used for filter conjugation. After conjugation, cells were serially diluted, and spotted on LB-antibiotic plates for measuring colony forming units (cfu). Exconjugants were selected by two selective markers (RifR TetR), which prevents growth of donor and target bacteria in the mixed cell suspension. LB plates containing Rif were used to calculate the total number of recipient cells (see, e.g., FIG. 1A). Next, the conjugation efficiency using the conjugative plasmid, pCON4-45, was tested. pCON4-45 is a derivative of RK2, which has a deletion of the 6kb NsiI-AsiSI fragment including the IS21 and the Par/Mrs region on RK2. This deleted region is not essential for conjugation of this plasmid. Thus, pCON4-45 is a self-transmissible plasmid. After filter conjugation, cells were serially diluted for plating on Rif and Rif/Tet plates (see, e.g., FIG. 1B). Colonies were counted on both plates and efficiency of conjugation was calculated.

### Example 4

### Conjugation and Killing Efficiencies of pCON15-56A

The non self-transmissible killer plasmid pCON15-56A (see, FIG. 5) was constructed as described in Example 2. The conjugation and the killing efficiencies of the plasmid were monitored using E. coli as a recipient/target. A regular filter conjugation was used to monitor the efficiency of conjugation (see, Example 3 and FIG. 1B). To monitor conjugation efficiency, an E. coli strain carrying the immE3 gene was used to neutralize the incoming toxin gene to prevent the recipient from being killed.

Donor bacterium carrying both pCON15-56A and pCON1-94 can secrete active ColE3 toxin into the culture medium, and kill neighboring ColE3-sensitive bacteria. The complex of ColE3 and its immunity protein ImmE3 form a complex, and secrete outside of the donor bacteria. This complex binds to an E. coli surface receptor (James et al., Microbiology 142 1569-1580 (1996)), the toxin is translocated into the cell, and cell death ensues. In the process of filter conjugation, both donor and recipient/target cells are mixed, and the colicin-sensitive recipient/target can be killed with the secreted toxin around the donor cells in a conjugation-independent manner. However, when a mutation takes place in this receptor, E. coli strains carrying such mutations no longer are killed by ColE3 because the toxin can not be translocated into the cell. A mutant such as this (e.g., E. coli containing a mutation in the ColE3 receptor) was used as recipients to distinguish the conjugation-dependent killing from the conjugation-independent killing. This mutant E. coli strain was designated RL315-E3R, and is also a derivative of K12.

Conjugation and killing efficiency of the killer plasmid was then tested. The filter conjugation method (as described in Example 3) was used to mediate conjugation. After the conjugation, the mixture of the donor and the recipient cells were harvested, and serially diluted. The serially diluted cell suspensions were spotted on a set of LB plates containing rifampicin/tetracycline to selectively grow exconjugants. Specifically, donor cells (con4-11c/pCON15-56A/pCON1-94) were conjugated to two different E. coli strains: one is sensitive to the killer plasmid (RL315-E3R), and the other one is resistant (RL315-E3R/pCON1-94). The resistant strain carries the helper plasmid with immE3 and so are protected from the colE3 on the killer plasmid. After filter conjugation, mixture of the donor and the recipient cells were serially diluted, and spotted on a Rif/Tet plate on which only exconjugants can grow. Column ‘a’ shows the results with the ColE3-sensitive strain as a recipient, and Column ‘b’ shows the results with the ColE3- resistant strain as a recipient.

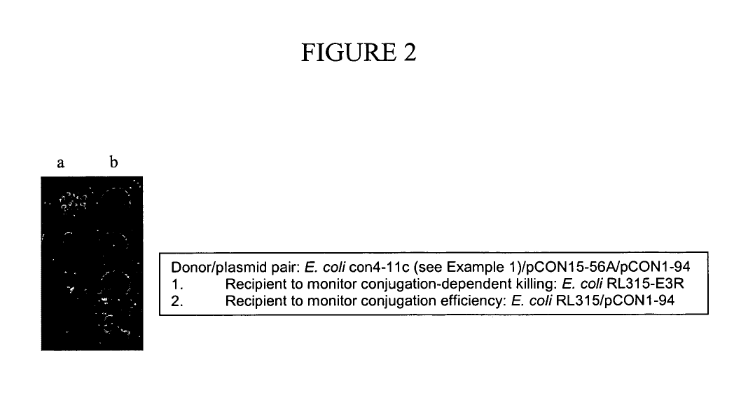

The survival of the resistant strain shows that the killer plasmid is successfully transferred into the recipient strains. Thus, the lack of growth in of the sensitive strains indicates that these cells were killed by the expression from the transferred ColE3 gene, rather than by the selective growth medium (see FIG. 2. From top to bottom, dilutions were as follows: x1, x10⁻², x10⁻⁴ and x10⁻⁶).

Approximately 55 and 6×10⁶ growing colonies were counter from the killer plasmid or non-killer plasmid treated recipient, respectively (see, e.g., FIG. 2). The comparison of these two numbers demonstrates that the survival of exconjugants treated with the killer plasmid was approximately 0.001%. Thus, using the compositions described herein, the present invention provides a very efficient and effective method of terminating target bacterial cells.

### Example 5

### In vivo Efficacy Testing

Using the donor/plasmid pair described in Example 4, in vivo efficacy was tested using a murine burn/sepsis model. Briefly, the experimental animals received a third degree full thickness (15% total body surface area) dorsal scald bum by immersion in 100° C. water for 9 seconds. P. aeruginosa PA14 was then applied topically to the bum wound. This strain has been shown to be virulent to a number of hosts including plants, worms and animals (Rahme et al., Science 268, 1899-1902 (1995)). The amount of the pathogen was adjusted to 2×10⁴ cfi calculated according to its OD₆₀₀. Different amounts of donor cells comprising plasmid were applied immediately afterwards, and survival of the animals monitored.

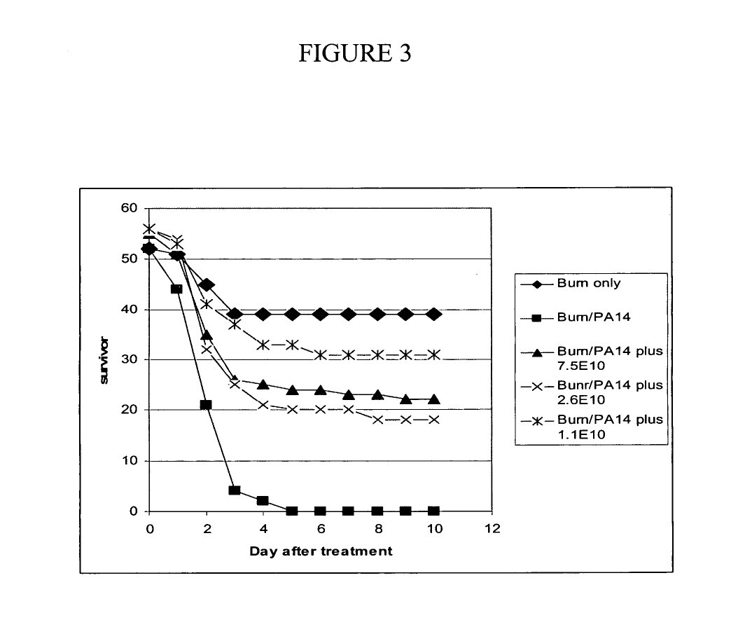

Nearly all mice exposed to PA14 alone were deceased after three days and all had died by day five (see, e.g., FIG. 3). However, mice that were exposed to PA14 and to donor bacterial cells comprising the killer plasmids had significantly reduced mortalities. On day 10, the percentage of surviving animals was compared between treatments and P value calculated. All P values were less than 0.000001, indicating the differences between the untreated mice (i.e., bum plus pathogen only) and treated mice (i.e., bum plus pathogen plus all doses of the donor bacterial cells) are highly significant.

### Example 6

### Construction of a New Killer Plasmid

RSF1010 is a mobilizable plasmid belonging to the IncQ group. The plasmids in this group can be conjugatively mobilized using a number of conjugation systems including RK2 (Lessl et al., J Bacteriol 174, 2493-2500 (1992)). When RK2 and RSF1010 and coexist in a single bacterium, the conjugation machinery provided by RK2 mobilizes RSF1010 very efficiently. Due to less-dependency on host bacterial factors for replication, this plasmid conjugates P. aeruginosa very efficiently, and approaches 100% efficiency frequently in the filter conjugation assay described in Example 3. Both RSF1010 and RK2 were combined to generate a self-transmissible plasmid to efficiently conjugate P. aeruginosa.

Specifically, the RK2-derived tra genes were combined with the backbone of RSF1010 to generate pCON19-79. The oriT sequence from RK2 was mutagenized to prevent the transfer of the plasmid from this region. The plasmid replication function of RK2 was abolished by deletion of RK2-derived origin of replication oriV. Instead, pCON19-79 utilizes RSF1010-derived oriT and oriV for the mobilization and replication of the plasmid, respectively. colE3 is under the control of lacPO promoter, and its leaky expression is further inhibited by tandemly placed transcriptional terminators in front of this plasmid (see, e.g., Anthony et al., J Microbiol Methods 58, 243-250 (2004)).

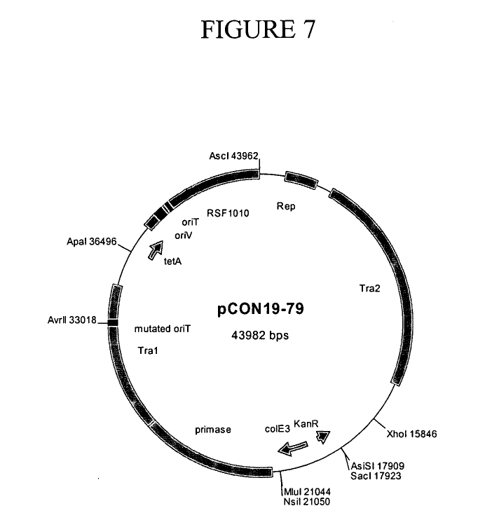

Expression of the tra gene on RK2 is finely tuned by a set of repressor proteins encoded on its own plasmid (Bingle et al., Mol Microbiol 49, 1095-1108 (2003)). Without these repressors, constitutive expression of the tra genes from the plasmid becomes lethal to the host cell, presumably due to formation of pores in the bacterial cell envelope (Grahn et al., J Bacteriol 182, 1564-74 (2000)). We call the feature of this repressor-less conjugative plasmid leading a high-level of constitutive tra expression CDC (Constitutively De-repressed Conjugation). The structure of this plasmid is depicted in FIG. 7.

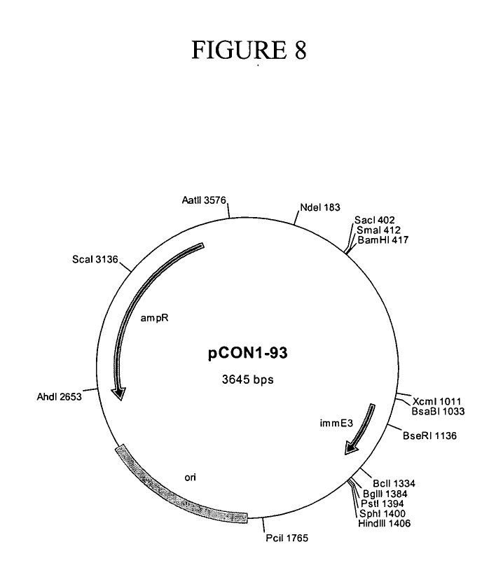

pCON1-93 (see, FIG. 8) was designed to maximize recipient/target killing using the combination of a killer gene and robust plasmid transmission. This plasmid can be maintained in an E. coli donor (e.g., con4-11c, See Example 1) along with a helper plasmid pCON1-93. The helper plasmid carries immE3 encoding the immunity protein for colicin E3, and the structure of this plasmid is shown in FIG. 8. The backbone of pCON1-93 was derived from pUC19, and immE3 was amplified by PCR, and cloned into the plasmid. immE3 is under the control of a constitutive promoter Pneo (promoter for neomycin-resistance determinant). These two plasmids, pCON19-79 and pCONl-93, were maintained in an E. coli donor CON4-11c (Example 1), and used for in vitro killing experiments.

### Example 7

### Demonstration of Improved in vitro Killing With a New Plasmid

The new killer plasmids developed as part of the present invention (e.g., See Example 6) were tested for killing capabilities. In addition to the new plasmids discussed herein, a new assay was designed in order to demonstrate the improved killing ability of the plasmids of the present invention (e.g., the plasmids pCON19-79 and pCON1-93 of Example 6). In this example, two different P. aeruginosa strains were used, and one Acinetobacter baumannii strain. Both P. aeruginosa strains were clinically isolated strains. One of them was an isolate from a wound patient, and shown to be resistant to multiple antibiotics (PanR: Poly-Antibiotic Resistance). A. baumannii is associated with bums and/or wounds, and often is found to be resistant to many clinically useful antibiotics, and therefore is becoming a major health threat. Both P. aeruginosa strains were rifampicin resistant, but the A. baumannii strain was not. As described in Example 2, a proper selective marker(s) is required to monitor conjugation efficiency, and rifampicin resistance was used to selectively grow recipient strains. Rifampicin-resistance mutants were obtained by spontaneous mutations on the chromosomal DNA. Briefly, overnight grown A. baumannii culture was spread on LB plates containing rifampicin, and growing mutants on these plates were isolated for the following experiment. Overnight-grown bacterial cultures of target strains were overlaid on the surface of LB plate containing rifampicin.

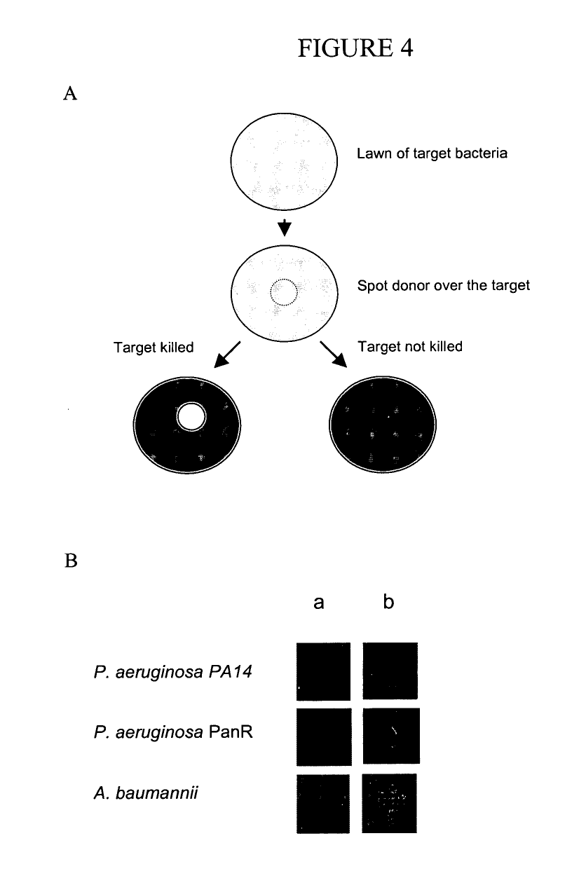

The donor bacterium carrying a killer plasmid (e.g., plasmids of Example 6) was grown overnight, serially diluted, and spotted over the lawn of the target bacteria. Only the recipient/target bacteria and exconjugants can selectively grow on the LB plate containing rifampicin. If the killer plasmids mobilize and kill the recipient cells efficiently, the area where the donor was spotted stays clear, leaving growth inhibitory zones. The strategy of this experiment is illustrated in FIG. 4A.

Briefly, a cell suspension of donor bacteria was spotted on the surface of target bacteria that are evenly spread over a LB plate containing rifampicin. In the presence of rifampicin only the target bacteria can grow. When the target cells were killed by the donor bacteria, the area where the cell suspension was spotted was left clear because the growth of both the donor and the target bacteria was prevented. If the donor does not have effect on (e.g., if the donors do not kill or attenuate growth of the recipient/target bacteria) the target bacteria the spotted area becomes covered by the growing target cells. Thus, using this assay, the efficacy of the newly constructed donor/plasmid pair on the three pathogens could be tested (see, e.g., FIG. 4B).

Each pathogen was treated with both a non-killer plasmid (treatment ‘a’) and a killer plasmid (treatment ‘b’). As seen in FIG. 4B, the killer plasmids (i.e., pCON19-79 generated in Example 6) formed growth inhibitory zones over the lawn of the pathogens (visible as darkened spots in FIG. 4B), evidencing the ability of the plasmid to kill the target bacteria. It is noted that the donor bacteria secrete small amounts of colicin E3 into the culture medium. However, each of the pathogens tested in this experiment are not sensitive to the toxin in the culture medium, presumably due to their lack of the receptor for this toxin on the cellular surface.

### Example 8

### In vivo Efficacy Testing with pCON19-79

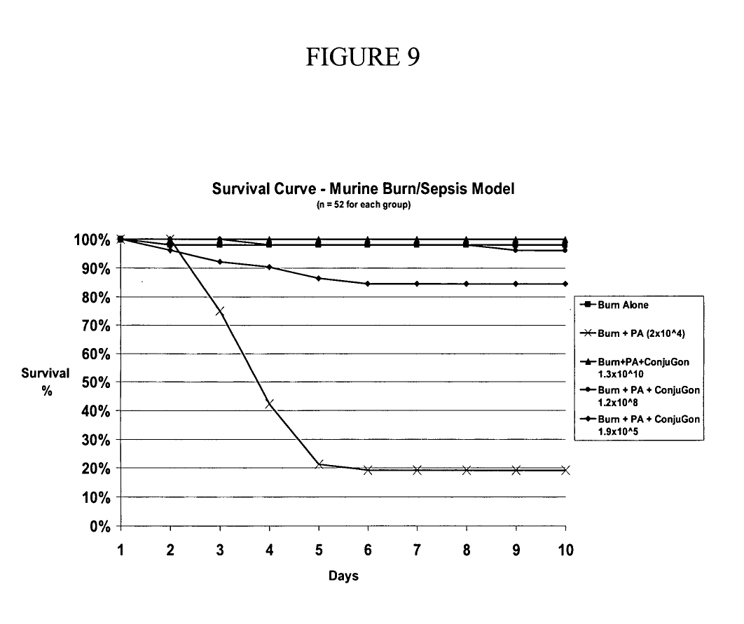

Experiments similar to those performed in Example 5 were performed with the plasmid pCON19-79 generated in Example 6. Briefly, experimental animals received a third degree 12% TBSA (total body surface area) dorsal scald burn by immersion in 85° C. water for 9 seconds. Pseudomonas aeruginosa PA14 was then applied topically to the burn wound. Immediately following application of PA1 4, donor cells carrying pCON19-79 were applied to the burn surface at various doses. Survival of the mice was monitored for 10 days. 42 out of 52 control mice receiving Pseudomonas aeruginosa PA14 at 2×10⁴ cfu without application of donor cells comprising pCON19-79 had died within six days after application of Pseudomonas aeruginosa PA14 to the burn (See Table 1, below). However, mice receiving Pseudomonas aeruginosa PA14 at 2×10⁴ cfti plus various doses of donor cells comprising the pCON19-79 plasmid displayed remarkably improved survival rates compared to the controls (see, e.g., FIG. 9). For example, of the 52 mice receiving Pseudomonas aeruginosa PA14 at 2×10⁴ cfu and 1.3×10¹⁰ cfu of donor cells, none had died as far out as 10 days post application. Significant improvements in mortality rates were also observed when lower doses of donor cells were used (see, e.g., FIG. 9).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of treating a tissue, comprising:
exposing a tissue to a donor cell, wherein said donor cell comprises:
i) a recombinant transmissible plasmid comprising a gene encoding a bactericidal protein; and
ii) a helper plasmid comprising a gene encoding an immunity protein, wherein said immunity protein is configured to inhibit said bactericidal protein;
wherein said donor cell is configured to conjugatively transfer said recombinant transmissible plasmid to a recipient cell, and wherein said recombinant transmissible plasmid is configured to express said gene encoding a bactericidal protein in said recipient cell.

2. The method of claim 1, wherein expression of said gene encoding a bactericidal protein is lethal to said recipient cell.

3. The method of claim 1, wherein said bactericidal protein is a colicin.

4. The method of claim 3, wherein said colicin is colE3.

5. The method of claim 1, wherein said bactericidal protein is selected from the group consisting of colA, colB, colD, colIa, colIb, colK, colN, colE1, colE2, colE4, colE5, colE6, colE7, colE8, colE9, and lysozyme.

6. The method of claim 1, wherein said immunity protein binds to said bactericidal protein.

7. The method of claim 4, wherein said immunity protein is immE3.

8. The method of claim 1, wherein said immunity protein is selected from the group consisting of colicin A, colicin B, colicin D, colicin Ia, colicin Ib, colicin K, colicin N, colicin E1, colicin E2, colicin E4, colicin E5, colicin E6, colicin E7, colicin E8, and colicin E9 immunity proteins.

9. The method of claim 1, wherein said treating of said tissue comprises treating a wound in said tissue.

10. The method of claim 9, wherein said wound comprises a bum wound.

11. The method of claim 1, wherein said tissue is in contact with a recipient cell prior to the exposing of said tissue to said donor cell.

12. The method of claim 1, wherein said tissue is not in contact with a recipient cell prior to the exposing of said tissue to said donor cell.

13. The method of claim 1, wherein said tissue is skin.

14. The method of claim 1, wherein said recombinant transmissible plasmid is self-transmissible.

15. The method of claim 1, wherein said recombinant transmissible plasmid is selected from the group consisting of pCON15-56A, pCON19-79.

16. The method of claim 1, wherein said helper plasmid is selected from the group consisting of pONI-93 and pCON1-94.

17. The method of claim 1, wherein said recipient cell comprises a bacterial cell.

18. The method of claim 17 wherein said bacterial cell is a pathogenic bacterial cell.

19. The method of claim 18 wherein said bacterial cell is of a genus selected from the group consisting of Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, or Chlamydiae.

20. A composition comprising a donor cell, said donor cell comprising:
i) a recombinant transmissible plasmid comprising a gene encoding a bactericidal protein; and
ii) a helper plasmid comprising a gene encoding an immunity protein, wherein said immunity protein is configured to inhibit said bactericidal protein;
wherein said donor cell is configured to conjugatively transfer said recombinant transmissible plasmid to a recipient cell, wherein said recombinant transmissible plasmid is configured to express said gene encoding a bactericidal protein in said recipient cell.

21. The composition of claim 20, wherein said bactericidal protein is a colicin.

22. The composition of claim 21, wherein said colicin is colE3.

23. The composition of claim 20, wherein said bactericidal protein is selected from the group consisting of colA, colB, colD, colia, colIb, colK, colN, colE1, colE2, colE4, colE5, colE6, colE7, colE8, colE9, and lysozyme.

24. The composition of claim 20, wherein said immunity protein binds to said bactericidal protein.

25. The composition of claim 22, wherein said immunity protein is immE3.

26. The composition of claim 20, wherein said immunity protein is selected from the group consisting of colicin A, colicin B, colicin D, colicin Ia, colicin Ib, colicin K, colicin N, colicin E1, colicin E2, colicin E4, colicin E5, colicin E6, colicin E7, colicin E8, and colicin E9 immunity proteins.

27. The composition of claim 20, wherein said transmissible plasmid comprises oriT and oriV of RSF1010, and wherein said gene encoding ColE3 is under control of a lac promoter/operator.

28. The composition of claim 20, wherein said gene encoding an immunity protein is under control of a promoter, wherein said promoter is constitutively active.

29. The composition of claim 28, wherein said promoter is Pneo.

30. The composition of claim 20, wherein said gene encoding an immunity protein is under control of a promoter, wherein said promoter is inducible.

31. The composition of claim 20, wherein said transmissible plasmid is pCON15-56A or pCON19-79.

32. The composition of claim 20, wherein said helper plasmid is pCON1-93 or pCON1-94.

33. A method of treating a surface comprising providing to said surface a composition comprising a donor cell, wherein said donor cell comprises:
i) a recombinant transmissible plasmid comprising a gene encoding a bactericidal protein; and
ii) a helper plasmid comprising a gene encoding an immunity protein, wherein said immunity protein is configured to inhibit said bactericidal protein;
wherein said donor cell is configured to conjugatively transfer said recombinant transmissible plasmid to a recipient cell, wherein said recombinant transmissible plasmid is configured to express said gene encoding a bactericidal protein in said recipient cell.

34. The method of claim 33, wherein said surface is present on one or more of a medical device, a wound care device, a body cavity device, a human body, an animal body, a personal protection device, a birth control device, and a drug delivery device.

35. The method of claim 33, wherein said surface comprises silicon, plastic, glass, polymer, ceramic, photoresist, skin, tissue, nitrocellulose, hydrogel, paper, polypropylene, cloth, cotton, wool, wood, brick, leather, vinyl, polystyrene, nylon, polyacrylamide, optical fiber, natural fibers, nylon, metal, rubber or composites thereof.

36. The method of claim 33, wherein said treating inhibits growth of recipient cells on said surface.

37. The method of claim 36, wherein said treating kills recipient cells that come into contact with said surface.

38. The method of claim 33, wherein said plasmid is self-transmissible.

39. A method of treating a surface comprising:
a) providing a composition comprising a donor cell, wherein said donor cell comprises one or more plasmids selected from the group consisting of pCON15-56A, pCON19-79, pCON1-93 and pCON1-94; and
b) exposing said composition to said surface.
